(19) United States
(12) Patent Application Publication
(10) Pub. No.: US 2024/0100394 A1
(43) Pub. Date: Mar. 28, 2024

(54) EVALUATION METHOD AND APPARATUS FOR STRENGTH TRAINING

(71) Applicant: CHENGDU FIT-FUTURE TECHNOLOGY CO., LTD., Chengdu (CN)

(72) Inventor: Yihong SHEN, Chengdu (CN)

(73) Assignee: CHENGDU FIT-FUTURE TECHNOLOGY CO., LTD., Chengdu (CN)

(21) Appl. No.: 18/045,526

(22) Filed: Oct. 11, 2022

(30) Foreign Application Priority Data
Sep. 22, 2022 (CN) 202211156837.6
Sep. 22, 2022 (CN) 2022111568376

(51) Int. Cl. A63B 24/00; A63B 71/06

(52) U.S. Cl. CPC A63B 24/0062; A63B 71/0622; A63B 2024/0068; A63B 2071/063; A63B 2220/30

(57) ABSTRACT

Disclosed are an evaluation and apparatus for strength training. The method includes: acquiring a standard action amplitude when a user performs an action for strength training; acquiring a training action amplitude when the user performs the action; and acquiring an action quality determination result by comparing the training action amplitude and the standard action amplitude corresponding to the action.

### CROSS-REFERENCE TO RELATED APPLICATIONS

This application claims priority to Chinese Patent Application No. 202211156837.6, filed on Sep. 22, 2022, the entire contents of which are hereby incorporated by reference.

### TECHNICAL FIELD

The present disclosure relates to the field of exercises, and in particular, relates to an evaluation method and apparatus for strength training.

### BACKGROUND

With the improvement of living standard, maintaining healthy physical state has become the basic pursuit of people. However, in actual life, people are confused about how to choose the right exercise or fitness programs to achieve their goals. Therefore, smart exercise apparatuses that are capable of making exercise plans according to users' own situations have broad market prospects.

Effective smart exercise hardware not only needs to a user an exercise plan and support the user to perform training, but also needs to monitor the quality of the user's activities to ensure that the user's activities are as standard as possible, such that the user achieves a better exercise effect. In addition, when a user performs a set of actions, each action in the set of actions has a relatively large difference in performance over another, and the user cannot adapt to the formulated intensity of actions. This may degrade user experience, and hinders long-term use of the exercise apparatus.

At present, an action feedback mechanism of the smart exercise apparatus covers fewer actions and lacks detailed evaluation of action quality, and fewer scenes and dimensions may be covered in this mechanism. The feedback mechanism fails to reflect all dimensions of user actions. Further, when giving action feedback, the accuracy is low, and thus this feedback mechanism is not convenient for long-term use.

### SUMMARY

Embodiments of the present disclosure provide an evaluation method and apparatus for strength training, which provide action feedback in multiple dimensions, and the information fed back is more scientific and reasonable, facilitating long-term use of the exercise apparatus.

According to one aspect of the embodiments of the present disclosure, an evaluation method for exercise training is provided. The method includes: in strength training by a user, recording a distance by which a cable is pulled out for performing a first action of a first set of actions regarding to a same action program, and acquiring a standard action amplitude when the user performs the action; acquiring a training action amplitude when the user performs the same action; and acquiring an action quality determination result by comparing the training action amplitude and the standard action amplitude corresponding to the same action.

According to the embodiments of the present disclosure, a standard action amplitude is defined, and an action amplitude of the action performed by the user each time is compared with the standard action amplitude to determine whether the action of the user is qualified when the user performs the action. Compared with the conventional methods, the technical solutions according to the present disclosure may be applicable to all the actions of training performed using a pull cable rope, and thus the application scope is more extensive. Further, by the comparison between the training action amplitude and the standard action amplitude, the quality of each action is evaluated, the evaluation results are more accurate. In addition, the evaluation method is applicable to different users, and the settings are more scientific and reasonable.

Further, for better evaluation on details of the action quality, the standard action amplitude includes a centripetal standard action amplitude and a centrifugal standard action amplitude, wherein the centripetal standard action amplitude is a distance by which a cable is pulled out centripetally for performing a first action of a first set of actions regarding a same action program, and the centrifugal standard action amplitude is a distance by which the cable is pulled out centrifugally for performing the first action of the first set of actions regarding the same action program. Further, the training action amplitude includes a centripetal action amplitude and a centrifugal action amplitude, wherein the centripetal action amplitude is a centripetal pull-out distance by which a cable is pulled out centripetally for performing a same action, and the centrifugal action amplitude is a centrifugal pull-out distance by which the cable is pulled out centrifugally for performing the same action. In strength training, the user's centripetal action amplitude and centrifugal action amplitude are evaluated separately to ensure the accuracy and details of each motion evaluation.

In some exemplary embodiments, the method further includes: acquiring a centripetal motion start point from which the cable is pulled out centripetally in a positive speed direction and at a speed greater than or equal to V, wherein V represents a predetermined first speed value; acquiring a centripetal motion end point Ki to which the cable is pulled centripetally in the positive speed direction and at a speed less than V within a first consecutive time, wherein the centripetal motion start point one-to-one corresponds to the centripetal motion end point, and each time a centripetal motion end point is generated, a distance from the centripetal motion start point to the centripetal motion end point is calculated; and calculating distances from all centripetal motion start points to their corresponding centripetal motion end points, and acquiring the centripetal pull-out distance.

In some exemplary embodiments, the method further includes: acquiring a centrifugal motion start point from which the cable is pulled out centrifugally in a negative speed direction and at a speed greater than or equal to V, wherein V represents a predetermined first speed value; and acquiring a centrifugal motion end point from which the cable is pulled out centrifugally in the negative speed direction and at a speed less than Z within a second consecutive time, wherein Z represents a predetermined second speed value, wherein the centrifugal motion start point one-to-one corresponds to the centrifugal motion end point, and each time a centrifugal motion end point is generated, a distance from the centrifugal motion start point to the centrifugal motion end point is calculated; and calculating distances from all centrifugal motion start points to their corresponding centrifugal motion end points, and acquiring the centrifugal pull-out distance.

The centripetal pull-out distance and the centrifugal pull-out distance are both greater than or equal to a predetermined distance.

In some exemplary embodiments, acquiring the action quality determination result includes: determining the action quality determination result as an incorrect action in the case that the training action amplitude is greater than the standard action amplitude; determining the action quality determination result as an unqualified action in the case that the training action amplitude is greater than or equal to a first predetermined ratio of the standard action amplitude and less than a second predetermined ratio of the standard action amplitude, wherein the first predetermined ratio is less than the second predetermined ratio; determining the action quality determination result as a qualified action in the case that the training action amplitude is greater than or equal to a second predetermined ratio of the standard action amplitude and less than a third predetermined ratio of the standard action amplitude, wherein the second predetermined ratio is less than the third predetermined ratio; and determining the action quality determination result as an incorrect action in the case that the training action amplitude is less than the first predetermined ratio of the standard action amplitude.

Further, for better acknowledgment of the training details for the user in strength training, according to the present disclosure, a corresponding action feedback is output based on the action quality determination result, wherein the action feedback includes, but is not limited to, page animation or voice prompt. In this way, the user is capable of better acquiring the training information.

In some exemplary embodiments, the method further includes: each time a centripetal motion end point is generated, acquiring a current accumulated centripetal motion distance by accumulating the distances from all the centripetal motion start points to their corresponding centripetal motion end points; and comparing the current accumulated centripetal motion distance with the corresponding standard action amplitude, acquiring the action quality determination result, and outputting a corresponding action feedback based on the action quality determination result.

According to another aspect of the embodiments of the present disclosure, an electronic device is further provided. The electronic device includes a memory, a processor, and one or more computer programs that are stored on the memory and runnable on the processor. The processor, when loading and running the one or more computer programs, is caused to perform the steps of the evaluation method for strength training as described above.

According to another aspect of the embodiments of the present disclosure, a non-transitory computer-readable storage medium is further provided. The computer-readable storage medium stores one or more computer programs. The one or more programs, when loaded and run by a processor, cause the processor to perform the steps of the evaluation method for strength training as described above.

The one or more technical solutions according to the embodiments of the present disclosure at least achieve the following technical effects or advantages:

The technical solutions according to the present disclosure cover all dimensions of actions of the user, and fine evaluation on the action quality of the user is achieved. The technical solutions are more widely used, achieve a high accuracy, and are suitable for long-term application.

### BRIEF DESCRIPTION OF THE DRAWINGS

The accompanying drawings herein, which are incorporated herein and constitute a part of the specification, illustrate several exemplary embodiments of the present disclosure, construing no limitation to the present disclosure. In the drawings:

FIG. 1 is a schematic diagram of a centripetal motion start point k to a centripetal motion end point k and a centrifugal motion start point k to a centrifugal motion end point k;

FIG. 2 is a schematic diagram of a centripetal motion distance feedback and a centrifugal motion distance feedback; and

FIG. 3 is a schematic diagram of a structure of an exercise apparatus according to an exemplary embodiment of the present disclosure.

### DETAILED DESCRIPTION

For better and clearer understanding of the objects, features, and advantages of the present disclosure, the present disclosure is described in detail with reference to the attached drawings and specific embodiments. It should be noted that in cases of no conflict, the embodiments and features in the embodiments of the present disclosure may be combined together.

The description hereinafter illustrates more details for better understanding of the present disclosure. However, the present disclosure may also be implemented by various embodiments different from those described herein. Therefore, the protection scope of the present disclosure is not limited by exemplary embodiments described hereinafter.

A person skilled in the art may understand that in the description of the present disclosure, the terms “longitudinal,” “lateral,” “upper,” “lower,” “front,” “rear,” “left,” “right,” “vertical,” “horizontal,” “top,” “bottom,” “inner,” “outer,” and the like indicate orientations or positional relationships which are based on the illustrations in the accompanying drawings, and these terms are merely for ease and brevity of the description, instead of indicating or implying that the devices or elements shall have a particular orientation and shall be structured and operated based on the particular orientation. Accordingly, these terms shall not be construed as limiting the present disclosure.

It may be understood that the article “an,” “a,” or “the” is interpreted as “at least one” or “one or more.” That is, in an embodiment, with respect to an element, one such element may be provided, whereas in other embodiments, a plurality of such elements may be provided. Therefore, use of the article ““an,” “a,” or “the” shall not be understood as a limitation to the number of elements. For example, in the description of the embodiments of the present disclosure, the term “multiple” or “a plurality of” signifies “at least two,” unless otherwise specified. Likewise, the term “a plurality of groups” or “multiple groups” signifies “at least two groups.”

The term “and/or” is merely an association relationship for describing associated objects, which represents that there may exist three types of relationships, for example, A and/or B may represent three situations: only A exists, both A and B exist, and only B exists. In addition, the symbol “/” generally represents an “or” relationship between associated objects before and after the symbol.

The method according to the embodiments of the present disclosure is applicable to a smart exercise apparatus provided with a motor and a pull assembly. The motor supplies a resistance force to the pull assembly. The pull assembly may be a pull cable. The motor supplies a resistance force against pull-out of the pull cable, and a user performs exercises by the pull cable with a resistance. Alternatively, the pull assembly may also be an arm. The motor supplies a resistance force against motion of the arm. The user performs training by the arm.

An exemplary embodiment of the present disclosure provides an evaluation method for strength training. The method includes: when a user performs strength training, recording a distance by which the cable is pulled out for performing a first action of a first set of actions regarding the same action, and defining the distance as a standard action amplitude when the user performs the action; the standard action amplitude includes a centripetal standard action amplitude and a centrifugal standard action amplitude, wherein the centripetal standard action amplitude is a distance by which the cable is pulled out centripetally for performing the first action of the first set of actions regarding the same action, and the centrifugal standard action amplitude is a distance by which the cable is pulled out centrifugally for performing the first action of the first set of actions regarding the same action; and acquiring a training action amplitude when the user performs the same action, wherein the training action amplitude includes a centripetal action amplitude and a centrifugal action amplitude, wherein the centripetal action amplitude is a centripetal pull-out distance by which a cable is pulled out centripetally for performing a same action program, and the centrifugal action amplitude is a centrifugal pull-out distance by which the cable is pulled out centrifugally for performing the same action.

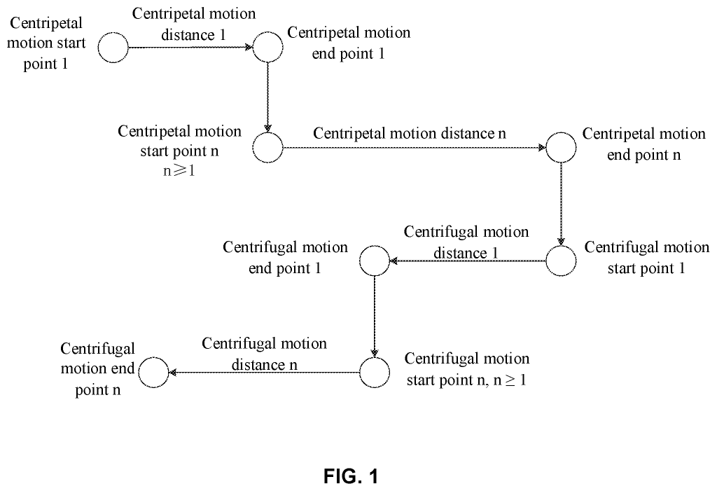

In some exemplary embodiments, acquiring the distance by which the cable is pulled out centripetally includes: acquiring a centripetal motion start point Si from which the cable is pulled out centripetally in a positive speed direction and at a speed greater than or equal to V, wherein V represents a predetermined first speed value, for example, V=2 cm/s, i∈\acute{\check{a}}\acute{\check{a}}[1, n], and n is a positive integer; acquiring a centripetal motion end point Ki to which the cable is pulled centripetally in a positive speed direction and at a speed less than V within a first consecutive time T1, for example, the consecutive time T1 is 100 ms, i∈[1, n], and n is a positive integer, wherein the centripetal motion start points Si one-to-one correspond to the centripetal motion end points Ki, and each time a centripetal motion end point Ki is generated, a distance from the corresponding centripetal motion start point Si to the corresponding centripetal motion end point Ki is calculated, as illustrated in FIG. 1; and calculating distances from all centripetal motion start points Si to their corresponding centripetal motion end points Ki, and acquiring a centripetal pull-out distance, where i∈[1, n], and n is a positive integer.

In some exemplary embodiments, acquiring the distance by which the cable is pulled out centrifugally includes: acquiring a centrifugal motion start point Hi from which the cable is pulled out centrifugally in a negative speed direction and at a speed greater than or equal to V, wherein i∈[1, n], and n is a positive integer; acquiring a centrifugal motion end point Fi to which the cable is pulled centrifugally in a negative speed direction and at a speed less than Z within a second consecutive time T2, for example, the consecutive time T2 is 200 ms, Z=1 cm/s, i∈[1, n], and n is a positive integer, wherein the centrifugal motion start points Hi one-to-one correspond to the centrifugal motion end points Fi, and each time a centrifugal motion end point Fi is generated, a distance from the corresponding centrifugal motion start point Hi to the corresponding centrifugal motion end point Fi is calculated, as illustrated in FIG. 1; and calculating distances from all centrifugal motion start points Hi to their corresponding centrifugal motion end points Fi are calculated, and acquiring a centrifugal pull-out distance, where i∈[1, n], and n is a positive integer.

In some exemplary embodiments, acquiring the action quality determination result includes: determining the action quality determination result as an incorrect action in the case that the training action amplitude is greater than the standard action amplitude; determining the action quality determination result as an unqualified action in the case that the training action amplitude is greater than or equal to a first predetermined ratio of the standard action amplitude and less than a second predetermined ratio of the standard action amplitude, wherein the first predetermined ratio is less than the second predetermined ratio, for example, the first predetermined ratio is 50%, and the second predetermined ratio is 85%; determining the action quality determination result as a qualified action in the case that the training action amplitude is greater than or equal to a second predetermined ratio of the standard action amplitude and less than a third predetermined ratio of the standard action amplitude, wherein the second predetermined ratio is less than the third predetermined ratio, for example, the third predetermined ratio is 100%; determining the action quality determination result as an incorrect action in the case that the training action amplitude is less than the first predetermined ratio of the standard action amplitude; and acquiring an action quality determination result by comparing the training action amplitude and the standard action amplitude corresponding to the same action.

In addition, a corresponding action feedback is output based on the action quality determination result, wherein the action feedback includes, but is not limited to, a page animation or a voice prompt.

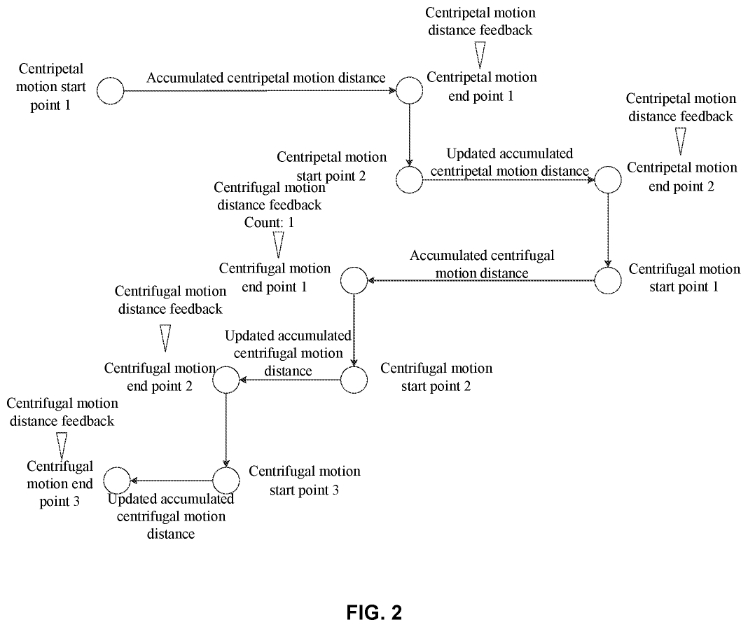

In some exemplary embodiments, as illustrated in FIG. 2, each time a centripetal motion end point Ki is generated, a current accumulated centripetal motion distance by accumulating the distances from all the centripetal motion start points Si to their corresponding centripetal motion end points Ki is acquired; and the action quality determination result is acquired by comparing the current accumulated centripetal motion distance with the corresponding standard action amplitude, and a corresponding action feedback is output based on the action quality determination result.

In some exemplary embodiments, the method further includes: acquiring a standard action amplitude when a user performs an action for strength training; acquiring the centripetal action amplitude and the centrifugal action amplitude of the action each time when a user performs the same action; comparing the centripetal action amplitude with the centripetal standard action amplitude, and comparing the centrifugal action amplitude with the centrifugal standard action amplitude; and prompting the completion quality of the actions to the user based on the action quality determination result, and outputting the feedback according to scores of different actions, wherein each determination result has a predetermined determination score, and the determination scores of different actions are displayed on an exercise apparatus.

In some exemplary embodiments, the exercise apparatus further includes a display configured to display exercise content (pre-recorded videos or live streams) and an interface that enables a user to personalize exercises. In addition, the smart exercise mirror allows a user and/or a trainer to interact with each other during exercises in a manner similar to conventional exercises performed in an exercise room or small exercise studio where the user and the trainer are in the same room (for example, providing a feedback to the trainer about the exercise rhythm, and correcting the user's form during a particular exercise program). The exercise apparatus is a smart exercise mirror, and may also include a processor configured to partially control operations of the various subcomponents in the smart exercise mirror, and manage data streams (for example, video content, audios from a trainer or user, and biometric feedback analysis) to/from the smart exercise mirror. The smart exercise mirror may also include a display for displaying video content, a graphical user interface with which a user is capable of interacting and controlling the smart exercise mirror, and a mirror including a reflective area. An image displayed by the display via the reflective area is superimposed with an image reflected by the user via the reflective area.

Details are listed in Table 1.

TABLE 1

| | Determination | | |
|---|---|---|---|
| Condition | result | Feedback | Action score |
| The training action amplitude is | Incorrect | The action amplitude | 0 |
| greater than the standard action | action | is over-great, and | |
| amplitude. | | the range of motion | |
| | | needs to be | |
| | | controlled. | |
| The training action amplitude is | Unqualified | The action is not | Single action = X * Y |
| greater than or equal to a first | action | qualified, and the | X = the training |
| predetermined ratio of the standard | | range of motion | action |
| action amplitude and less than a | | needs to be | amplitude/the |
| second predetermined ratio of the | | increased. | standard action |
| standard action amplitude. | | | amplitude |
| | | | Y = the standard |
| | | | score |
| | | | corresponding to |
| | | | the single action |
| The training action amplitude is | Quantified | Null | Single action |
| greater than or equal to a second | action | | score = Y |
| predetermined ratio of the standard | | | Y = the standard |
| action amplitude and less than a | | | score |
| third predetermined ratio of the | | | corresponding to |
| standard action amplitude. | | | the single action |
| The training action amplitude is | Incorrect | The action amplitude | 0 |
| less than the first predetermined | action | is over-small, and | |
| ratio of the standard action | | the action needs to | |
| amplitude. | | be completed to your | |
| | | best. | |

In some exemplary embodiments, in a set of deadlift actions, the user completes one set including nine deadlifts with a burden of centripetal 30 Kg and centrifugal 50 Kg. In the initial training of the user, the understanding and strength of the training action are weak, as listed in Table 2.

TABLE 2

| Scenario | 1 | 2 | 3 | 4 | 5 | 6 | 7 | 8 | 9 |
|---|---|---|---|---|---|---|---|---|---|
| Last feedback | Qualified | Unqualified | Incorrect | Qualified | Unqualified | Incorrect | Qualified | Unqualified | Incorrect |
| for centrifugal | | | | | | | | | |
| motion | | | | | | | | | |
| First feedback | Qualified | Qualified | Qualified | Incorrect | Incorrect | Incorrect | Unqualified | Unqualified | Unqualified |
| for centrifugal | | | | | | | | | |
| motion | | | | | | | | | |
| Comprehensive | Qualified | Unqualified | Incorrect | Incorrect | Incorrect | Incorrect | Unqualified | Unqualified | Incorrect |
| feedback | | | | | | | | | |
| Single action | 100 | 75 | 0 | 0 | 0 | 0 | 80 | 65 | 0 |
| score (the | | | | | | | | | |
| standard score | | | | | | | | | |
| corresponding | | | | | | | | | |
| to the single | | | | | | | | | |
| action Y = 100) | | | | | | | | | |

In the process of performing training, the user acquires a full score for a qualified action at the first time; at the second time, the action amplitude fail to reach the standard, the system prompts that the action amplitude is not up to the standard, and the user acquires a score of 75%; then at the following several times, the user constantly makes mistakes in strength control, continuously acquires low scores and error prompts.

In settlement a single course, the total score of the user is calculated by adding the total score of each set of actions. By comparing the user's total score with the full score, a course completion evaluation is acquired, and the evaluation indicates the training quality of the user's single course.

In some exemplary embodiments, the centripetal standard action amplitude is a distance by which a cable is pulled out centripetally for performing a first action of a first set of actions regarding a same action program, and the centrifugal standard action amplitude is a distance by which the cable is pulled out centrifugally for performing the first action of the first set of actions regarding the same action program. In addition, the standard action amplitude also satisfies the following requirements:
A maximum value pos_max(1) and a minimum value pos_recycle(1) that are respectively pulled and recycled by a user for a first time, and a maximum value pos_max(2) and a minimum value pos_recycle(2) that are respectively pulled and recycled by the user for a second time are acquired,
wherein pos_max (1) represents a final centripetal motion end point when the cable is pulled out for the first time, pos_recycle (1) represents a final centrifugal motion end point when the cable is recycled for the first time, pos_max (2) represents a final centripetal motion end point when the cable is pulled out for the second time, and pos_recycle (2) represents a final centrifugal motion end point when the cable is recycled for the second time;
in the case that pos_max(2)≥pos_max(1)−M and pos_max(2)−pos_recycle(2) M, the range of motion is calibrated as ROM=pos_max(1)−pos_recycle(1), wherein M is a predetermined first threshold, for example, M=0.05 m; and
in the case that pos_max(2)<pos_max(1)−M or pos_max(2)−pos_recycle(2)<M, the cable is pulled again until pos_max(n) pos_max(n−1)−M and pos_max(n)−pos_recycle(n) M, and then the range of motion is calibrated as ROM=pos_max(n−1)−pos_recycle(n−1).

That is, a centripetal pull-out distance and a centrifugal pull-out distance are both greater than a predetermined distance M, for example, M=5 cm.

In some exemplary embodiments, the exercise apparatus includes: a motor, a differential, arms, a pull assembly, and corresponding controllers, circuits and accessories. For example, the pull assembly includes pull cables, and a belt is connected between an output shaft of the motor and the differential. One end of the each of the pull cables is connected to the differential, and the other end of each of the pull cables is connected to a corresponding pull ring or other exercise accessories upon traveling along the arm. A user may perform exercises by pulling the pull cables during exercises, and may also perform exercises by the arms. The pull cables drive the motor to move via the differential and the belt. When the motor is powered on, an output torque, namely, a resistance force, is generated. When the user pulls the pull cables, the output torque of the motor needs to be overcome. In this way, the purpose of strength training is realized.

When performing exercises, the user inputs his/her own parameters on the exercise apparatus, including height, exercise level, training target, and the like, and selects different exercise modes during exercising. Upon selection of the exercise mode, the exercise apparatus acquires a predetermined weight according to the user parameters and the selected exercise mode, and adjusts the pull force output by the motor under a predetermined linear function corresponding to the exercise mode according to user's pulling. In this way, the user performs exercises more scientifically and safely and achieve a better exercise effect.

In some exemplary embodiments, the action feedback is displayed via a graphical user interface (GUI). The GUI includes page animation, which includes, but is not limited to, changing animation, highlighting or flashing a page icon involved in performing the action feedback, displaying a progress bar on the page information involved. The GUI further includes identifying the action being performed by the user and displaying a prompt action or a suggested action or the like when performing action control. In addition, the GUI further includes generating a completion prompt according to a voice instruction of the user and the like when performing voice control.

In some exemplary embodiments, a control element of the motor is capable of acquiring an output force value, a pull amplitude of the pull cable may be determined according to the number of turns of the motor, and the pull amplitude of the pull cable may be also measured by other devices such as sensors, namely, the training action amplitude is acquired.

The standard action amplitude may be predetermined by initialization by the user, or the standard action amplitude corresponding to the action may be acquired according to user parameter information.

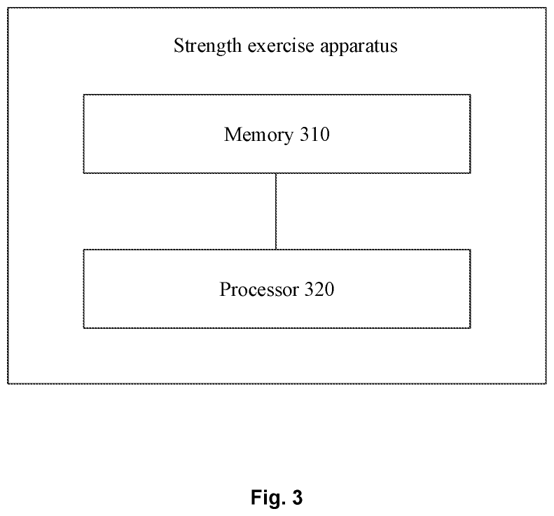

An exemplary embodiment of the present disclosure provides a strength exercise apparatus. FIG. 3 illustrates an electronic part of the strength exercise apparatus. The apparatus includes a memory 310, a processor 320, and one or more computer programs that are stored in the memory and executable on the processor. The one or more computer programs, when loaded and run by the processor, cause the processor to implement the steps of the evaluation method for strength training.

The processor may be a central processing unit (CPU), or may be a general purpose processor, a digital signal processor (DSP), an application specific integrated circuit (ASIC), a field-programmable gate array (FPGA), or a programmable logic device, a discrete gate or transistor logic device, a discrete hardware component, or the like, for example, a micro controller unit or the like. The general processor may be a microprocessor or any customary processor or the like.

The memory may be configured to store computer programs and/or modules, and the processor, when loading and running the computer programs and/or modules stored in the memory, is caused to implement the functions of the exercise apparatus according to the present disclosure. The memory may include a program memory area and data memory area, wherein the program memory area may store operation systems and application programs (for example, an audio play function, an image play function and the like) needed by at least one function. In addition, the memory may include a high-speed random-access memory, and may further include: a non-volatile memory, for example, a hard disk, a memory, and a plug-in hard disk; a smart memory card; a secure digital card; a flash memory; at least one magnetic disk storage device; a flash memory device; or other volatile solid-state storage devices, for example, a random-access memory (RAM), a read-only memory (ROM), an erasable programmable read-only memory (EPROM), an electrically erasable programmable read-only memory (EEPROM), a programmable read-only memory (PROM), a magnetic memory, a disk, a disc, and the like. A RAM may include a static RAM or a dynamic RAM.

An exemplary embodiment of the present disclosure provides a non-transitory computer-readable storage medium. The non-transitory computer-readable storage medium stores one or more computer programs, wherein the one or more computer programs, when loaded and run by a processor, cause the processor to implement the functions of the steps of the evaluation method for strength training.

The non-transitory computer-readable storage medium according to the embodiments of the present disclosure may employ any combination of one or a plurality of computer-readable media. The non-transitory computer-readable medium may be a computer-readable signal medium or a computer-readable storage medium. The non-transitory computer-readable storage medium may be, but is not limited to, for example, electrical, magnetic, optical, electromagnetic, infrared or semiconductor system, apparatuses or devices, or any combination thereof. More specific examples (non-exhaustive enumeration) of the computer-readable storage medium includes: an electrical connection having one or a plurality of conducting wires, a portable computer magnetic disk, a hard disk, a RAM, a ROM, an ERROM, an optical fiber, a portable compact disc read-only memory (CD-ROM or flash memory), an optical storage device, a magnetic storage device, or any combination thereof. In this specification, the computer-readable storage medium may be any tangible medium including or storing one or more programs. The one or more programs may be run by an instruction execution system, apparatus or device, or may be used by means of incorporation therewith.

The units or modules of the above embodiments, if implemented in the form of software functional units and sold or used as stand-alone products, may be stored in a computer-readable storage medium. Based on such an understanding, the technical solutions of the present disclosure essentially, or the part contributing to the common practices, or all or part of the technical solutions may be implemented in a form of a software product. The computer software product is stored in a storage medium and includes several instructions to cause a processor to perform all or some of steps of the methods described in the embodiments of the present disclosure. The storage medium includes various media capable of storing program code, for example, a USB flash disk, a removable hard disk, a ROM, a RAM, a magnetic disk, or an optical disc.

Although the exemplary embodiments of the present disclosure are described above, once knowing the basic creative concept, a person skilled in the art can make other modifications and variations to these embodiments. Therefore, the appended claims are intended to be construed as covering the exemplary embodiments and all the modifications and variations falling within the scope of the present disclosure.

Obviously, a person skilled in the art can make various modifications and variations to the present disclosure without departing from the spirit and scope of the present disclosure. Thus, it is intended that the present disclosure cover the modifications and variations of the present disclosure provided they come within the scope of the appended claims and their equivalents.

What is claimed is:

1. An evaluation method for strength training, comprising:
acquiring a standard action amplitude when a user performs an action for strength training;
acquiring a training action amplitude when the user performs the action; and
acquiring an action quality determination result by comparing the training action amplitude and the standard action amplitude corresponding to the action.

2. The evaluation method for strength training according to claim 1, further comprising:
outputting a corresponding action feedback based on the action quality determination result.

3. The evaluation method for strength training according to claim 1, wherein the standard action amplitude comprises a centripetal standard action amplitude and a centrifugal standard action amplitude, wherein the centripetal standard action amplitude is a distance by which a cable is pulled out centripetally for performing a first action of a first set of actions regarding a same action program, and the centrifugal standard action amplitude is a distance by which the cable is pulled out centrifugally for performing the first action of the first set of actions regarding the same action program.

4. The evaluation method for strength training according to claim 1, wherein the training action amplitude comprises a centripetal action amplitude and a centrifugal action amplitude, wherein the centripetal action amplitude is a centripetal pull-out distance by which a cable is pulled out centripetally for performing a same action, and the centrifugal action amplitude is a centrifugal pull-out distance by which the cable is pulled out centrifugally for performing the same action.

5. The evaluation method for strength training according to claim 3, further comprising:
acquiring a centripetal motion start point from which the cable is pulled out centripetally in a positive speed direction and at a speed greater than or equal to V, wherein V represents a predetermined first speed value; and
acquiring a centripetal motion end point to which the cable is pulled centripetally in the positive speed direction and at a speed less than V within a first consecutive time,
wherein the centripetal motion start point one-to-one corresponds to the centripetal motion end point, and each time a centripetal motion end point is generated, a distance from the centripetal motion start point to the centripetal motion end point is calculated; and
calculating distances from all centripetal motion start points to their corresponding centripetal motion end points, and acquiring the centripetal pull-out distance.

6. The evaluation method for strength training according to claim 3, further comprising:
acquiring a centrifugal motion start point from which the cable is pulled out centrifugally in a negative speed direction and at a speed greater than or equal to V, wherein V represents a predetermined first speed value; and
acquiring a centrifugal motion end point from which the cable is pulled out centrifugally in the negative speed direction and at a speed less than Z within a second consecutive time, wherein Z represents a predetermined second speed value,
wherein the centrifugal motion start point one-to-one corresponds to the centrifugal motion end point, and each time a centrifugal motion end point is generated, a distance from the centrifugal motion start point to the centrifugal motion end point is calculated; and
calculating distances from all centrifugal motion start points to their corresponding centrifugal motion end points, and acquiring the centrifugal pull-out distance.

7. The evaluation method for strength training according to claim 3, wherein the centripetal pull-out distance and the centrifugal pull-out distance are both greater than or equal to a predetermined distance.

8. The evaluation method for strength training according to claim 7, wherein acquiring the action quality determination result comprises:
determining the action quality determination result as an incorrect action in the case that the training action amplitude is greater than the standard action amplitude;
determining the action quality determination result as an unqualified action in the case that the training action amplitude is greater than or equal to a first predetermined ratio of the standard action amplitude and less than a second predetermined ratio of the standard action amplitude, wherein the first predetermined ratio is less than the second predetermined ratio;
determining the action quality determination result as a qualified action in the case that the training action amplitude is greater than or equal to the second predetermined ratio of the standard action amplitude and less than a third predetermined ratio of the standard action amplitude, wherein the second predetermined ratio is less than the third predetermined ratio; and
determining the action quality determination result as an incorrect action in the case that the training action amplitude is less than the first predetermined ratio of the standard action amplitude.

9. The evaluation method for strength training according to claim 2, wherein the action feedback comprises a page animation or a voice prompt.

10. The evaluation method for strength training according to claim 5, further comprising: each time a centripetal motion end point is generated, acquiring a current accumulated centripetal motion distance by accumulating the distances from all the centripetal motion start points to their corresponding centripetal motion end points; and comparing the current accumulated centripetal motion distance with the corresponding standard action amplitude, acquiring the action quality determination result, and outputting a corresponding action feedback based on the action quality determination result.

11. An exercise apparatus, comprising:
at least one processor;
and one or more memories coupled to the at least one processor and storing programming instructions for execution by the at least one processor to perform operations comprising:
acquiring a standard action amplitude when a user performs an action for strength training;
acquiring a training action amplitude when the user performs the action; and
acquiring an action quality determination result by comparing the training action amplitude and the standard action amplitude corresponding to the action.

12. The exercise apparatus according to claim 11, wherein the operations further comprise: outputting a corresponding action feedback based on the action quality determination result.

13. The exercise apparatus according to claim 11, wherein the standard action amplitude comprises a centripetal standard action amplitude and a centrifugal standard action amplitude, wherein the centripetal standard action amplitude is a distance by which a cable is pulled out centripetally for performing a first action of a first set of actions regarding a same action program, and the centrifugal standard action amplitude is a distance by which the cable is pulled out centrifugally for performing the first action of the first set of actions regarding the same action program.

14. The exercise apparatus according to claim 11, wherein the training action amplitude comprises a centripetal action amplitude and a centrifugal action amplitude, wherein the centripetal action amplitude is a centripetal pull-out distance by which a cable is pulled out centripetally for performing a same action, and the centrifugal action amplitude is a centrifugal pull-out distance by which the cable is pulled out centrifugally for performing the same action.

15. The exercise apparatus according to claim 13, wherein the operations further comprise:
acquiring a centripetal motion start point from which the cable is pulled out centripetally in a positive speed direction and at a speed greater than or equal to V, wherein V represents a predetermined first speed value; and
acquiring a centripetal motion end point to which the cable is pulled centripetally in the positive speed direction and at a speed less than V within a first consecutive time, wherein the centripetal motion start point one-to-one corresponds to the centripetal motion end point, and each time a centripetal motion end point is generated, a distance from the centripetal motion start point to the centripetal motion end point is calculated; and
calculating distances from all centripetal motion start points to their corresponding centripetal motion end points, and acquiring the centripetal pull-out distance.

16. The exercise apparatus according to claim 13, wherein the operations further comprise:
acquiring a centrifugal motion start point from which the cable is pulled out centrifugally in a negative speed direction and at a speed greater than or equal to V, wherein V represents a predetermined first speed value; and
acquiring a centrifugal motion end point from which the cable is pulled out centrifugally in the negative speed direction and at a speed less than Z within a second consecutive time, wherein Z represents a predetermined second speed value,
wherein the centrifugal motion start point one-to-one corresponds to the centrifugal motion end point, and each time a centrifugal motion end point is generated, a distance from the centrifugal motion start point to the centrifugal motion end point is calculated; and
calculating distances from all centrifugal motion start points to their corresponding centrifugal motion end points, and acquiring the centrifugal pull-out distance.

17. The exercise apparatus according to claim 13, wherein the centripetal pull-out distance and the centrifugal pull-out distance are both greater than or equal to a predetermined distance.

18. The exercise apparatus according to claim 17, wherein the operations further comprise:
determining the action quality determination result as an incorrect action in the case that the training action amplitude is greater than the standard action amplitude;
determining the action quality determination result as an unqualified action in the case that the training action amplitude is greater than or equal to a first predetermined ratio of the standard action amplitude and less than a second predetermined ratio of the standard action amplitude, wherein the first predetermined ratio is less than the second predetermined ratio;
determining the action quality determination result as a qualified action in the case that the training action amplitude is greater than or equal to the second predetermined ratio of the standard action amplitude and less than a third predetermined ratio of the standard action amplitude, wherein the second predetermined ratio is less than the third predetermined ratio; and
determining the action quality determination result as an incorrect action in the case that the training action amplitude is less than the first predetermined ratio of the standard action amplitude.

19. The exercise apparatus according to claim 12, wherein the action feedback comprises a page animation or a voice prompt.

20. The exercise apparatus according to claim 15, wherein the operations further comprise: each time a centripetal motion end point is generated, acquiring a current accumulated centripetal motion distance by accumulating the distances from all the centripetal motion start points to their corresponding centripetal motion end points; and comparing the current accumulated centripetal motion distance with the corresponding standard action amplitude, acquiring the action quality determination result, and outputting a corresponding action feedback based on the action quality determination result.
